(19) United States
(12) Patent Application Publication
(10) Pub. No.: US 2006/0173302 A1
(43) Pub. Date: Aug. 3, 2006

(54) Medical detector that uses multiple detector heads spaced over a limited arc

(76) Inventor: Richard L. Conwell, Del Mar, CA (US)

Correspondence Address: FISH & RICHARDSON, PC, P.O. BOX 1022, MINNEAPOLIS, MN, 55440-1022, US

(21) Appl. No.: 11/235,660

(22) Filed: Sep. 26, 2005

Related U.S. Application Data
(60) Provisional application No. 60/612,750, filed on Sep. 24, 2004.

(51) Int. Cl. A61B 6/00; G01T 1/166

(52) U.S. Cl. 600/436; 250/363.04

(57) ABSTRACT

Separated medical detector heads for medical single particle imaging, are all located clustered together at one end of an arc, e.g., over 202.5° or 180° of arc. The entire patient can still be imaged, but the patient can enter and exit the imaging area without going between the heads.

This application claims priority from provisional application Ser. No. 60/612,750, filed Sep. 24, 2004.

### BACKGROUND

Medical imaging systems may use nuclear materials for the imaging. One such imaging system is single photon emission computed tomography, abbreviated as SPECT. Other medical imaging techniques may include other kinds of general nuclear medicine, positron emission tomography or PET, as well as magnetic resonance imaging. Each of these is referred to herein generically as nuclear, single particle, medical emissions.

Imaging systems of this type may be dependent on many variables including, but not limited to, patient demographics, selection of the collimator which is used for the photon emission, the kind of radiation detector which is used, and the uptake of the radiopharmaceutical in the patient. The different ways in which these variables are carried out may affect the image quality that is.

SPECT imaging attempts to produce a three-dimensional reconstruction of the intensity of the three-dimensional distribution of a photon emitter within the body of the subject being imaged. Typically the emitted photons are gamma-rays, but may be any electromagnetic radiation. SPECT back projects multiple projection images acquired at equal angular increments over at least 180° around the subject. One or more detector heads collect these projection images and produce a two-dimensional image of the emitted gamma rays.

Having multiple detector heads typically increases the camera system's efficiency. This increased efficiency can either be used to improve image quality or to reduce the time of the procedure.

Current multi-head SPECT cameras are known. A dual head configuration, as well as configurations with more than two heads, may space the detector heads completely around the patient, that is, over 360°, surrounding the patient. For a two head system, the heads may be configured to be 90° apart.

A three-headed system may typically space the heads up at 120° increments, again surrounding the patient. In this arrangement, one of the heads is always in substantially the posterior position relative to the patient.

### SUMMARY

The present application defines an apparatus formed of a plurality of detector heads, each detecting a nuclear single particle based medical emission, where the detector heads are fixed relative to one another in an arc like overall shape, and the detector heads define a first arc, each of the heads stays within an arc of 202.5°. A motion system may move the heads and/or the patient. In other embodiments, the heads can stay within a 180° arc.

### BRIEF DESCRIPTION OF THE DRAWINGS

In the drawings:

FIG. 1 shows a first embodiment where all heads are within 180°;

FIG. 2 shows a second embodiment, where all heads are within 202.5° of arc;

FIG. 3 shows a 4 head, 1800° arc embodiment; and

FIG. 4 shows a block diagram of the overall system.

### DETAILED DESCRIPTION

The general structure and techniques, and more specific embodiments which can be used to effect different ways of carrying out the more general goals are described herein.

The prior art configuration discussed above may not be optimal for cardiac single photon emission computed tomography, or SPECT imaging with Thallium-201, where collection over the posterior 180° of the patent is not necessarily efficient. In addition, having the heads arrayed over 360° makes patient ingress and egress from the imaging system more difficult. The patient can only enter the imaging volume by going between the heads into the imaging volume along the axis of rotation of the heads.

The overall embodiment is shown in FIG. 4. A rotational movement structure 400 causes either the heads or the patient or both, to rotate. This induces a movement between the heads and patient to enable complete viewing of the human body by the detector heads. The heads 410 may be in any of the configurations described herein. The heads produce outputs 420, which are processed by a controller 430, in a conventional way. The controller may also control the movement device 400, and may produce an output on the user interface 440.

One embodiment recognizes that it is only necessary to collect projection images over 180° of the object to be reconstructed. Collecting projections over only 180° can also reduce image time. In addition, for some procedures, such as human cardiac SPECT with Thallium-201, it may be desirable to collect over the anterior 180°, since collection over the posterior 180° may be less efficient due to attenuation.

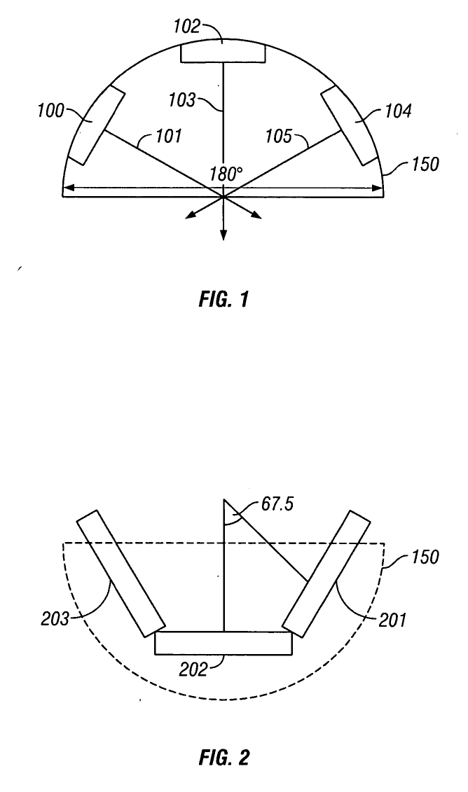

The present embodiment goes against the established teaching in the art by forming a system with multiple heads, either three heads, or more than three heads, clustered all together. According to one aspect of this system, the three heads are positioned inside an arc whose outer dimensions encompass 180°. The arc and head placement is shown for example in FIG. 1, where the three heads 100, 102, 104 are all positioned within the 180 degree arc 150.

According to another aspect, however, the physical size of the outer portions of the heads causes the heads to extend beyond the 180 degree arc. Therefore, an alternative aspect of this system is that axes defining the facing directions of the heads are all within the 180 degree arc 150. As shown in FIG. 1, the head 100 has an axis facing in the direction 101, the head 102 defines an axis facing in the direction 103, and the head 104 defines an axis facing in the direction 105. Each of these axes 101, 103, 105 is maintained within the 180 degree arc 150, even if the physical size of the head causes the head to extend beyond the 180° arc.

Another feature allows the heads and/or the axes to extend beyond the 180 degree arc, to an arc of approximately 200°, for example 202.5°, in the embodiment shown in FIG. 2. In this scenario, there may be a 67.5 degree angle between the heads 201 and 202, and correspondingly, a 135 degree angle between the heads 201, 203. Even though the physical size of the heads causes them to extend beyond the 180 degree arc, this may be considered an optimum arrangement. In this arrangement, there is 67.5° between the heads, and the patient also rotates by 67.5° in order to obtain a full imaging. Even with the 202.5 embodiment, there is still at least a 157.5° of angular arc where there is no detector heads, which allows patients ingress through that area.

Another feature is that all of the multiple heads are clustered together, with less than 5 degrees of angular separation between the heads.

Another embodiment describes an open design with three or more heads. This embodiment allows a multi-head Single Photon Emission Computed Tomography (SPECT) camera system to be constructed whereby three or more of the detector heads are positioned within an arc of 180°.

Recent imaging gamma ray detector heads have been developed that have small dead spaces surrounding the imaging field-of-view (FOV). In addition, small field-of-view (SFOV) detector heads that have an area just large enough to cover the area of the human heart may be used to perform cardiac SPECT. These two together are used to arrange three or more of these SFOV heads within an arc of 180°.

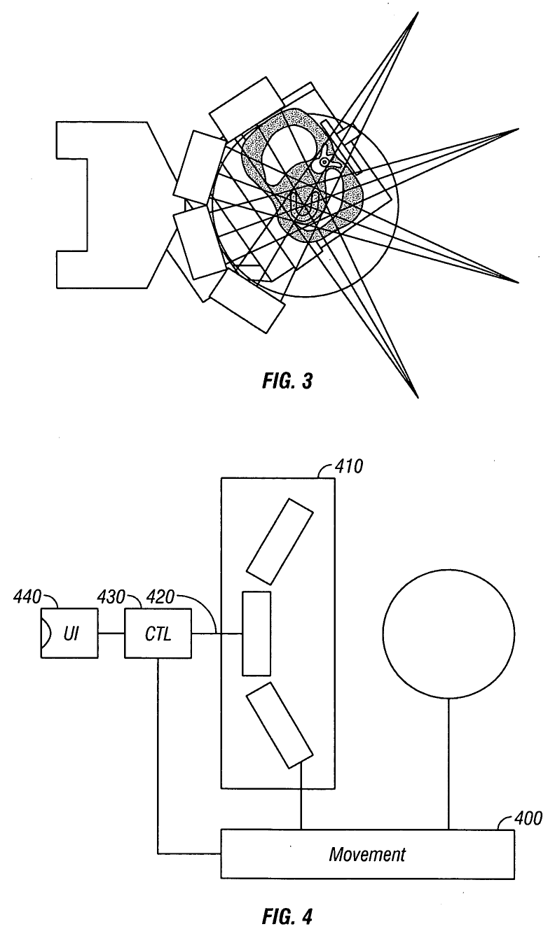

FIG. 3 illustrates an embodiment using four heads. The inventor does not intend for the embodiments to be limited to systems with three or four heads. Any number of heads can be used, e.g., 5, 6, 7 or any larger number of heads.

The example of cardiac SPECT imaging of a human has been used to describe the embodiment. However, the embodiment can be used to acquire brain SPECT data of a human or a whole body SPECT of appropriately sized animals. This embodiment is also usable with other medical imaging systems, such as PET and others.

Moreover, while FIG. 3 depicts the use of a fan-beam collimator, it should be understood that parallel hole collimators or collimators of other configurations can also be used. The figure also illustrates an upright, rotating chair gantry system whereby the subject rotates before the imaging heads. The imaging heads could also be affixed to a gantry that rotates the heads around the subject to be imaged. The subject may be upright, prone or supine.

Also, while specified angles are given herein, other angular configurations and other emissions can be used.

Although only a few embodiments have been disclosed in detail above, other embodiments are possible and the inventor intends these to be encompassed within this specification. The specification describes specific examples to accomplish a more general goal that may be accomplished in other way. This disclosure is intended to be exemplary, and the claims are intended to cover any modification or alternative which might be predictable to a person having ordinary skill in the art. For example, other numbers of heads, other imaging systems, and the like, may be used with this system.

Also, the inventor intends that only those claims which use the words “means for” are intended to be interpreted under 35 USC 112, sixth paragraph. Moreover, no limitations from the specification are intended to be read into any claims, unless those limitations are expressly included in the claims.

What is claimed is:

1. An apparatus, comprising:
a plurality of detector heads, each detecting a nuclear, single particle based medical emission, said detector heads fixed relative to one another in an arc like overall shape, and said detector heads defining a first part, each of which stays within 202.5° over said arc; and
a motion system, which moves to rotate information received by the plurality of detector heads.

2. An apparatus as in claim 1, wherein said detector heads are single photon emission computed tomography heads.

3. An apparatus as in claim 1, wherein said first aspect is an overall size of the head, and the entire detector heads are within said 202.5° arc.

4. An apparatus as in claim 1, wherein said first aspect is a pointing direction of the head, and said pointing direction is within said 202.5° arc.

5. An apparatus as in claim 1, wherein there are three of said detector heads.

6. An apparatus as in claim 1, wherein there are four of said detector heads.

7. An apparatus as in claim 1, wherein said motion system moves a patient relative to the detector heads.

8. An apparatus as in claim 1, wherein said motion system moves the detector head relative to the location of a patient.

9. An apparatus as in claim 1, wherein said heads stay within an arc of 180°.

10. An apparatus as in claim 1, wherein a spacing between each two adjacent heads is 5° of said arc or less.

11. A medical imaging apparatus, comprising: a plurality of detector heads, receiving information indicative of nuclear single particle based medical emissions, said plurality of detector heads being arranged to define a portion of an arc of a circle, clustered at one side of said arc, and leaving a first opening at an opposite side of said arc, where there is at least a 157.5° of angular arc where there is no detector heads.

12. An apparatus as in claim 10, wherein said detector heads are single photon emission detector heads.

13. An apparatus as in claim 10, wherein said detector heads are positron emission detector heads.

14. An apparatus as in claim 10, wherein said detector heads are located with their entire extent within said 202.5°, with the area outside their extent being completely open.

15. An apparatus as in claim 10, wherein said detector heads are located with their pointing directions being within said 202.5°.

16. An apparatus as in claim 10, wherein there are three detector heads, each of which have a 67.5° angle therebetween.

17. An apparatus as in claim 16, further comprising a part which rotates, to change the information received by the detector heads.

18. An apparatus as in claim 17, wherein said part rotates the detector heads.

19. An apparatus as in claim 17, wherein said part rotates a patient relative to the detector heads.

20. An apparatus as in claim 17, wherein said part rotates the information over substantially 67.5° to obtain a full imaging.

21. A method, comprising:
locating a plurality of separated medical detector heads over a portion of a continuous arc, that leaves an opening at an opposite side of the arc;
allowing the patient to enter in the opening; and
moving either one of the patient or the medical detector heads or both over an angle while obtaining medical images from the patient, to obtain a substantially complete image of the patient.

22. A method as in claim 21, further comprising using said medical heads to obtain a single photon emission as said medical images.

23. A method as in claim 21, wherein said detector heads do not extend outside of 202.5° of arc.

22. A method as in claim 21, wherein there are three detector heads, with a 67.5° angle therebetween.

23. A method as in claim 22, further comprising moving said at least one of the patient or medical heads over 67.5° during said obtaining.

24. A method as in claim 21, wherein said locating comprises locating at least three of said medical detector heads at different angles, but substantially abutting against one another.

25. A method as in claim 21, wherein said locating comprises locating at least three of said medical detector heads at different angles, but separated by 5° of separation or less.
